(19) United States
(12) Patent Application Publication
(10) Pub. No.: US 2024/0083078 A1
(43) Pub. Date: Mar. 14, 2024

(54) Method for Manufacturing a Mold Element for the Production of Micro-arrays and a Mold Element

(71) Applicants: Olga Grünwald, Koblenz (DE); Michael Kulik, Urmitz (DE)

(72) Inventors: Olga Grünwald, Koblenz (DE); Michael Kulik, Urmitz (DE)

(73) Assignee: LTS Lohmann Therapie-Systeme AG, Andernach (DE)

(21) Appl. No.: 18/275,694

(22) PCT Filed: Jan. 13, 2022
(86) PCT No.: PCT/EP2022/050670

§ 371 Date: Aug. 3, 2023

(30) Foreign Application Priority Data
Feb. 4, 2021 (DE) 10 2021 102 555.9

(51) Int. Cl. B29C 33/38; B29C 33/40; B29C 33/42; B29C 59/02; B29C 59/04

(52) U.S. Cl. CPC B29C 33/3878; B29C 33/40; B29C 33/42; B29C 59/026; B29C 59/046

(57) ABSTRACT

provided is a method for manufacturing a mold element for the production of microarrays, including the following steps: (i) providing a planar base element having a first surface and a second surface opposite the first surface, (ii) providing a planar auxiliary element on the second surface, (iii) penetrating the base element from the first surface in order to form mold openings, and (iv) reversibly or non-reversibly entering the auxiliary element when penetrating the base element. Moreover, a mold element for the production of microarrays, including a planar base element having a first surface a second surface opposite the first surface, a planar auxiliary element arranged on the second surface, and several mold openings extending from the surface of the base element through the second surface of the base element.

### CROSS-REFERENCE TO RELATED APPLICATIONS

This application is the United States national phase of International Application No. PCT/EP2022/050670 filed Jan. 13, 2022, and claims priority to German Patent Application No. 10 2021 102 555.9 filed Feb. 4, 2021, the disclosures of which are hereby incorporated by reference in their entireties.

### BACKGROUND OF THE INVENTION

### Field of the Invention

The invention relates to a method for manufacturing a mold element for the production of microarrays. The invention further relates to a mold element, in particular manufactured with the method according to the invention, for the production of microarrays.

### Description of Related Art

Microarrays have a plurality of microneedles that are typically arranged in a support element, such as a patch, a plaster or the like, or are connected to the same. Microarrays have a high number of microneedles, the length of which is dimensioned such that, when being pushed into the skin of a patient, they penetrate into the skin only so far that the needle tips do not come into contact with nerves and vessels, if possible. The needles comprise an active ingredient, for example, a medicament. The corresponding active ingredient may be provided on an upper side of the needles or be provided in the needles. When the active ingredient is arranged in the needles, the needles or components of the needles are made of a material that dissolves in the skin of a patient.

Microarrays are produced using silicone molds, for example, which have a plurality of mold openings formed as recesses and serving as a negative mold. In one variant to fill these recesses, a liquid provided with the active ingredient is typically applied onto the upper side of the silicone mold. After the liquid has dried, another liquid is applied, if necessary.

The silicone molds with their recesses are currently manufactured by means of an injection molding process, for example.

### SUMMARY OF THE INVENTION

It is an object of the invention to provide a method for manufacturing a mold element for the production of microarrays, which is cost-effective and preferably suitable for the production of high quantities. It is also an object of the invention to provide a corresponding mold element.

According to the invention, the object is achieved by a method as described herein and a mold element as described herein, respectively.

The method according to the invention for manufacturing a mold element for the production of microarrays is in particular a method for manufacturing an open mold element, in particular open on both sides, for the production of microarrays. In a first step of the method a planar base element is provided. The base element has a first surface and a second surface opposite to the first surface. The first surface is in particular the upper side of the base element, while the second surface is in particular the lower side of the base element. A further, second step is to provide a planar auxiliary element on the second surface. In particular, the step of providing the auxiliary element is performed before or after providing the base element, wherein it is however preferred to provide the auxiliary element together with the base element. After providing the base element and after providing the auxiliary element, the base element is penetrated in a third step from the first surface in order to form mold openings in the base element. Penetration here means in particular complete penetration of the base element so that the base element is completely penetrated. The mold openings formed by this procedure thus preferably extend from the first surface to the second surface so that continuous openings are formed. When penetrating the base element, there is also an entry into the auxiliary element. If the base element is penetrated by means of a punching tool, for example, the punching tool completely penetrates the base element and also penetrates the auxiliary element. It is particularly preferred that there is partial entry into the auxiliary element. Thus, it is hereby preferred that the auxiliary element is not completely penetrated. The entry is reversible or non-reversible. In other words, non-reversible means an invasive entry into the auxiliary element, so that the entry creates an open recess in the auxiliary element, in particular a permanent one. The non-reversible entry is thus preferably a transformative or separating entry. If the penetration is carried out by means of a punching tool, for example, the base element is penetrated with the punching tool and the punching tool also partially penetrates the auxiliary element so that a partial opening is also carried out here. In contrast, a reversible entry means, in other words, a non-invasive entry. Thus, it is particularly preferred that, in the case of a reversible entry, a deformation back, in particular after a penetration out of the auxiliary element, of the auxiliary element takes place at the point of entry. Thus, in particular, no opening remains at the penetrated point of the auxiliary element. It is particularly preferred that the auxiliary element is configured to be deformable and/or elastic so that a deformation back may take place via the elasticity. A further preferred step after penetrating the base element and entering the auxiliary element consists in penetrating out of the base element as well as penetrating out of the auxiliary element. Entering the base element and the auxiliary element is carried out together, preferably with the same tool. It is particularly preferred that penetration out of the base element and the auxiliary element is carried out together. If the penetration and entry is carried out by means of a punching tool, for example, penetrating out takes place when the punching tool is withdrawn. In particular, the mold openings extend through the base element and partially into the auxiliary element.

With the invention, the mold openings can be filled from both sides in an advantageous manner. Preferably, they can be filled from above and from below. This offers advantages such as shorter process times or the concentration of an active ingredient in the needle tip. The method according to the invention has advantages over an alternative method for manufacturing open structures, in which openings are produced in a laser process downstream of an injection molding process. Such a disadvantageous injection molding process as well as the laser process is very time-consuming and cost-intensive. In contrast, the method according to the invention is cost-effective and preferably suitable for the production of large quantities. This is realised in particular by the simplified process control and the combination of both processes, manufacturing and opening the mold openings, in one step.

In a preferred embodiment, the auxiliary element is connected to the base element. Preferably, the connection between the auxiliary element and the base element is configured to be detachable. It is particularly preferred that the connection is adhesive, in particular detachably adhesive. The planar auxiliary element is in particular connected with a planar side to the second surface of the base element. Advantageously, the detachable connection implements that the auxiliary element can be removed from the base element after forming the mold openings and thus the base element can be used as a molding device.

It is preferred that providing the base element and providing the auxiliary element is effected by jointly providing a composite element. Here, the composite element comprises the base element and the auxiliary element. It is particularly preferred that the composite element has a film composite, in particular consists thereof.

It is preferred that the penetration is carried out by embossing. The embossing is carried out with an embossing tool having in particular at least one, preferably a plurality of protrusions complementary to the mold openings. It is particularly preferred that the penetration is carried out by hot embossing. The embossing temperature is preferably 80-260° C.

The penetration, in particular the embossing, takes place continuously or discontinuously.

It is preferred that when penetration is carried out with the embossing tool, the entry is also carried out with the same embossing tool. By means of the auxiliary element, it is advantageously implemented in particular that during embossing, the embossing tool is protected from damage and/or wear, since it is shielded when entering the auxiliary element and thus cannot come into contact with other objects or the like that could lead to damage, for example.

Alternatively or in addition to the embossing penetration, it is possible that the penetration is carried out by means of drilling and/or milling and/or punching.

If the penetration is carried out by embossing, it is preferred that this is done by means of an embossing roller. If, however, punching is involved, it is preferred that this is done by means of a punching roller. It is also possible, for example, that embossing is carried out by means of an embossing die or punching is carried out by means of a punching die.

After the entry, in particular after a subsequent penetration out, a further step takes place: cooling. Preferably, at least the base element is cooled down. It is preferred that the auxiliary element is also cooled down. Preferably they are both cooled down to room temperature.

A further step of the method consists in the removal of the auxiliary element from the base element. Particularly preferred is pulling the auxiliary element off the base element. In particular, the step of removing the auxiliary element from the base element takes place after the step of entering, wherein it is particularly preferred that the step of removal takes place after cooling.

The mold element for the production of microarrays according to the invention has a planar base element. The base element has a first surface and a second surface opposite to the first surface. The first surface is preferably the upper side, while the second surface is preferably the lower side of the base element. A planar auxiliary element is arranged on the side of the second surface of the base element. Preferably, the auxiliary element is arranged on the second surface of the base element. Preferably, the auxiliary element is connected to the second surface of the base element. Furthermore, the mold element has several mold openings extending from the first surface of the base element through the second surface of the base element. Thus, the mold openings extend completely through the base element. In other words, the base element is thus continuously open on both sides. It is preferred that the mold openings are embossed mold openings. The mold openings are in particular negative molds for microarrays to be produced.

It is preferred that the mold openings extend partially into the auxiliary element. Thus, it is preferred that the mold openings extend from the first surface of the base element into a part of the auxiliary element. In particular, the mold openings do not extend completely through the auxiliary element.

In a preferred embodiment, the auxiliary element is connected to the base element. In particular, the connection between the auxiliary element and the base element is configured to be detachable. It is particularly preferred that the connection between the auxiliary element and the base element is adhesive, preferably detachable-adhesive.

In a preferred embodiment, the base element and/or the auxiliary element comprises thermoplastic and/or thermoplastic elastomer, in particular consists thereof. It is particularly preferred that the base element and/or the auxiliary element comprises TPU, PC, APET, PPC and/or PETG, in particular consists thereof.

It is preferred that the base element and/or the auxiliary element includes, in particular consists of a film. Preferably, the film has a thickness of 0.2-2.0 mm, particularly preferred 0.5-1.5 mm. It is particularly preferred that the base element has a film with a thickness of 0.5-1.5 mm and/or the auxiliary element has a film with a thickness of 0.1-2 mm.

It is preferred that the mold openings are cylindrical or conical in shape. If, for example, forming the mold openings is carried out by means of embossing, it is preferred that the embossing tool has one or more likewise cylindrical or conical protrusions complementary to the cylindrical or conical mold openings. The mold openings preferably correspond to negative molds of the microarrays to be produced. It is preferred that the base area of the cylindrical or conical mold opening is on the first surface of the base element. In particular, the base area of the cylindrical or conical mold opening is a circle, oval, rectangle or square. If the mold openings have a conical shape, they can also be truncated cones. The cross-section of the mold openings preferably tapers from the top side of the base element towards the bottom side. Preferably, the mold openings are symmetrical in the longitudinal direction, in particular rotationally symmetrical, so that the opening provided on the bottom side is arranged centrally with respect to a base area of the mold opening.

It is preferred that the auxiliary element, in particular the material from which the auxiliary element is made, has a higher hot forming temperature than the base element, in particular than the material from which the base element is made. Preferably, the auxiliary element has a higher melting temperature than the base element. It is preferred that the auxiliary element has a substantially higher hot forming temperature than the base element. Preferably, the auxiliary element has a higher elasticity than the base element. In particular, the auxiliary element has a smaller modulus of elasticity than the base element. The auxiliary element may have a porous structure, for example. It is possible that the auxiliary element has one or more of the features described in this paragraph.

Preferably, each individual mold opening has on the first surface a cross-sectional area of 0.04 mm²-0.16 mm², in particular of 0.04-0.08 mm². The mold openings themselves preferably have a depth of 600 μm to 2200 μm, in particular 600 μm to 1000 μm. It is preferred that the mold openings extend through the entire thickness of the base element. In particular, the mold openings extend into the auxiliary element with a depth of 10 μm to 500 μm, preferably with a depth of 50 μm to 300 μm, particularly preferably with a max. depth of 80 μm.

The base element, in particular in the form of a film, preferably has a thickness of 500 μm-1.5 mm.

The auxiliary element, in particular in the form of a film, preferably has a thickness of 100 μm-2 mm.

The mold openings on the second surface of the base element and/or the openings on the auxiliary element preferably have cross-sectional area of <1200 μm², in particular <100 μm². Alternatively or in addition, the mold openings on the second surface of the base element and/or the openings on the auxiliary element preferably have a diameter of <40 μm, in particular <10 μm.

The preferred composite element, which has at least the base element and the auxiliary element and which is designed in particular as a film composite, preferably has a thickness of 600 μm to 3.5 mm.

It is preferred that the auxiliary element can be detached, in particular pulled off, at least partially from the base element. It is possible that the auxiliary element is at least partially detached from the base element, in particular pulled off.

Preferably, the mold openings have a small distance and a high density, respectively. In particular, 9-350 mold openings are provided per square centimetre in a particularly regular arrangement. Likewise, it is possible to arrange mold openings in rows, for example, with the adjacent rows each being arranged on gap.

In a particularly preferred embodiment, the preferred size ratios described above are produced by the method described above, so that the corresponding features in a particularly preferred embodiment can also individually or in combination define the preferred embodiment of the method.

The above-described method according to the invention can preferably be supplemented by one or more features of the above-described mold element according to the invention. The above-described mold element according to the invention can preferably be supplemented by one or more features of the above-described method according to the invention.

### BRIEF DESCRIPTION OF THE DRAWINGS

In the following, the invention is described in more detail by means of a preferred embodiment with reference to the accompanying drawings.

In the Figures:

FIG. 1 is a schematic side view of a composite element having a base element and an auxiliary element,

FIG. 2 is a schematic side view of a composite element together with an embossing roller,

FIG. 3 is a schematic side view of an embossed composite element, and

FIG. 4 is a schematic detailed view of the section of FIG. 3 with a mold opening formed by the method according to the invention,

FIG. 5 is a schematic side view of a further composite element together with an embossing roller, and

FIG. 6 is a schematic side view of a further composite element together with an embossing roller.

### DESCRIPTION OF THE INVENTION

In the Figures, similar or identical components or elements are identified by the same reference numerals or variations thereof (e.g. 10 and 10′). In particular in the interest of improved clarity, preferably elements already identified are not provided with reference numerals in all Figures.

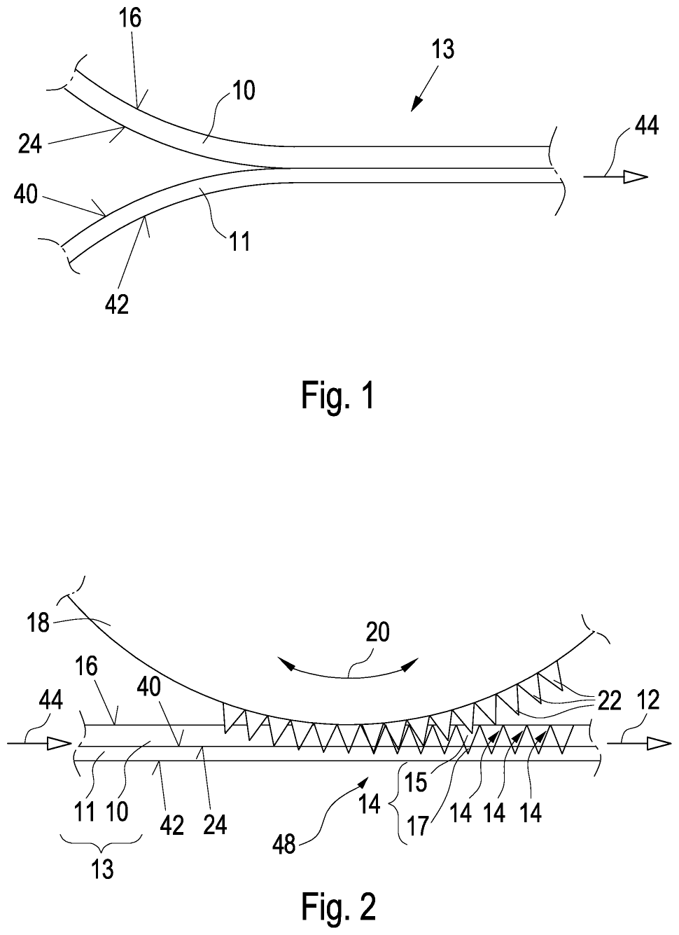

FIG. 1 is an example of a composite element 13 as a starting base for manufacturing a mold element with the aid of the method according to the invention. Composite element 13 comprises a base element 10 and an auxiliary element 11. In particular, base element 10 and auxiliary element 11 are films so that composite element 13 corresponds to a film composite. To form composite element 13, base element 10 and auxiliary element 11 are provided, as shown, wherein they are provided by being introduced from the left. A second surface side 24 of base element 10 is adhesively connected to a first surface 40 in order to form composite element 13. The adhesive connection is made in particular by means of adhesive arranged, for example, on the first surface 40 of auxiliary element 11 and/or on the second surface of base element 10. For example, the adhesive may be an adhesive layer connected to the first surface 40 of the auxiliary element and/or to the second surface 24 of base element 10. On the other hand, it is possible to create the adhesive connection through molecular forces of the film surfaces.

Shown on the right, auxiliary element 11 and base element 10 are adhesively connected to composite element 13 and are continued, shown by arrow 44.

Opposite the second surface 24 of base element 10 is the first surface 16 of base element 10. Opposite the first surface 40 of auxiliary element 11 is the second surface 42 of auxiliary element 11. Preferably, the first surface is a top side and/or the second surface is a bottom side.

For manufacturing a mold element with the aid of the method according to the invention, a composite element 13, in particular in the form of a film composite, is moved from the left (arrow 44) in the direction of arrow 12 in the exemplary embodiment shown in FIG. 2. Preferably, composite element 13 shown in FIG. 2 is composite 13 of FIG. 1.

To create mold openings 14 in a first surface 16 of base element 10, an embossing roller is preferably provided which rotates in the direction of an arrow 20, in the exemplary embodiment counterclockwise. A plurality of protrusions 22 are provided on an outside of the embossing roller 18. Regularly recurring areas of the outside of the embossing roller are provided with protrusions 22. Alternatively, it is possible that the protrusions 22 are regularly distributed over the entire outside embossing roller 18. As shown, the cross-section of the protrusions 22 corresponds to the cross-section of the mold openings 14. Shown here, the mold openings 14 extend completely through base element 10 and continue into a portion of auxiliary element 11. The portion of mold opening 14 extending through base element 10 corresponds here to a base mold opening 15, which preferably serves as a later die for producing the microarrays. The part of mold opening 14 in auxiliary element 11 represents an auxiliary opening 17. The protrusions 22 are in particular pyramid-shaped and have a preferably square or round cross-section.

The height of the protrusions 22 and thus the depth of the mold openings 14 is less than the thickness of composite element 13. Thus, during the embossing process, the protrusions 22 do not come into contact with an underground 48. Thus, the protrusions 22 are protected from damage and/or wear, in particular by impact with underground 48. It is also in particular advantageously implemented that the mold openings 14 to the bottom side shown are protected from the environment by auxiliary element 11 and are thus protected from contamination.

It is preferred that before or after filling the base mold openings 15, auxiliary element 11 is removed, in particular pulled off, from base element 10.

Composite element 13 extending in the direction of arrow 12 with embossed mold openings 14 corresponds to a mold element 100 to be manufactured. On the one hand, it is possible that to form mold element 100, auxiliary element 11 is connected to base element 10, or auxiliary element 11 is partially removed from base element 10, or auxiliary element 11 is completely removed from base element 10.

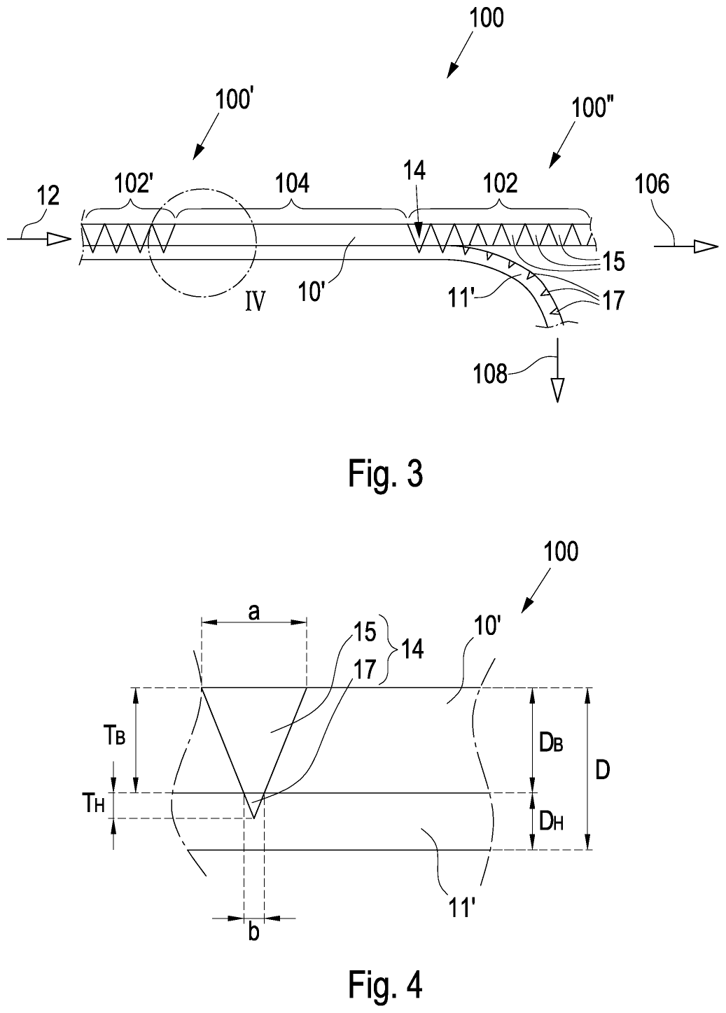

FIG. 3 is an exemplary embodiment of a mold element 100 according to the invention, which was preferably produced using an embodiment of the method according to the invention. It is preferred that mold element 100 of FIG. 3 is mold element 100 of FIG. 2. Mold element 100 is introduced from the left in the direction of arrow 12.

As shown, mold element 100 has regions 102, 102′ with embossed mold openings 14 and region 104 without embossed mold openings 14. Shown on the right side, auxiliary element 11′ is removed from base element 10′ in the direction of arrow 108, in particular pulled off. Base element 10′ continued in the direction of arrow 106 thus has the base mold openings 15, while the removed auxiliary element 11′ has the auxiliary recesses 17. As shown, base element 10′ with the base mold openings 15 and auxiliary element 11′, which is removed therefrom, with the auxiliary recesses 17 thus form mold element 10′. The illustrated mold element 100 thus corresponds to a first exemplary embodiment of a mold element according to the invention, such as a mold element with partially removed auxiliary element 11′. The mold element 100′ also illustrated corresponds to a second exemplary embodiment of a mold element to be manufactured, such as a mold element with a connected auxiliary element 11. The mold element 100″ also illustrated corresponds to a third exemplary embodiment of a mold element to be manufactured, such as a mold element with a completely removed auxiliary element 11′. In particular, these different embodiments of the mold element are different states and/or regions of a mold element according to the invention to be manufactured.

FIG. 4 is a detailed section of area IV of FIG. 3. A side length a of the in particular square or round base area of the pyramid-shaped mold opening has a dimension of preferably a=200-400 μm. The side length a of mold opening 14 preferably also corresponds to the side length of the base area of base mold opening 15. The side length b of the base area of the auxiliary recess 17 preferably has a dimension of b=3-20 μm. The depth of mold opening 14 is preferably t=600 μm-2.200 μm. The depth of auxiliary recess 17 is preferably t_{H}=50-500 μm. The depth of base mold opening 15 preferably t_{B}=550-2150 μm or 550-1700 μm. The thickness of composite element 13 is particularly d=800 μm-4 mm. The thickness dB of the base element is preferably 600 μm-2 mm. The thickness dB of the auxiliary element is preferably 200 μm-2 mm.

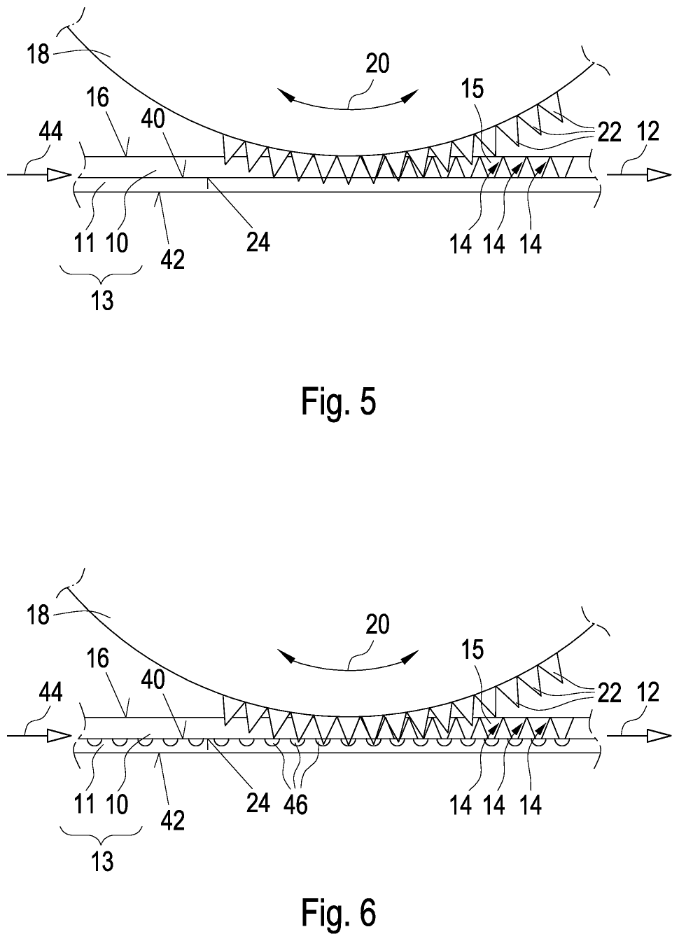

The exemplary embodiment of FIG. 5 substantially corresponds to the one of FIG. 2. In contrast to the embodiment of FIG. 2, in FIG. 6 auxiliary element 11 has a high elasticity and/or a high embossing temperature, so that after entry of the protrusions 22 into auxiliary element 11 no auxiliary recesses 17 (see FIG. 2) remain in auxiliary element 11, but instead auxiliary element 11 preferably forms back into its original shape. Accordingly, the mold openings 14 according to the exemplary embodiment of FIG. 5 are the base mold openings 15.

The exemplary embodiment of FIG. 6 substantially also corresponds to the exemplary embodiment of FIG. 2. In contrast to the exemplary embodiment of FIG. 1, auxiliary element 11 of FIG. 6 has a porous structure consisting of the recesses 46. The recesses 46 are, for example, pyramidal or cylindrical or part-circular in shape. The recesses 46 are preferably regularly distributed on the first surface of auxiliary element 11. The recesses 46 are oriented in such a way that during the embossing process, the tips of the protrusions 22 enter the recesses 46 and thus do not deform auxiliary element 11. Again, in the exemplary embodiment of FIG. 6, a mold opening 14 thus corresponds to a base mold opening 15.

1. A method for manufacturing a mold element for the production of microarrays, comprising the following steps:
providing a planar base element having a first surface and a second surface opposite the first surface,
providing a planar auxiliary element on the second surface,
penetrating the base element from the first surface in order to form mold openings, and
reversibly or non-reversibly entering the auxiliary element when penetrating the base element.

2. The method according to claim 1, wherein the auxiliary element is connected to the base element, in particular detachably, wherein it is particularly preferred that the auxiliary element is adhesively connected to the base element.

3. The method according to claim 1, wherein providing the base element and providing the auxiliary element is done by providing a composite element, preferably a film composite, comprising the base element and the auxiliary element.

4. The method according to claim 1, wherein the penetration is effected by embossing, in particular hot embossing, wherein the embossing is effected with an embossing tool having at least one, preferably a plurality of projections complementary to at least a part of the mold openings.

5. The method according to claim 4, wherein the embossing is carried out by means of an embossing roller or an embossing die.

6. The method according to claim 1, characterized by cooling the base element and preferably the auxiliary element after entry.

7. The method according to claim 1, characterized by removing, in particular pulling off the auxiliary element from the base element.

8. A mold element for the production of microarrays, in particular manufactured by the method according to claim 1, comprising
a planar base element having a first surface and a second surface opposite the first surface,
a planar auxiliary element arranged on the second surface, in particular connected to the second surface, and
several, in particular embossed mold openings extending from the first surface of the base element through the second surface of the base element.

9. The mold element according to claim 8, wherein the mold openings extend partially into the auxiliary element.

10. The mold element according to claim 8, wherein the auxiliary element is connected to the base element, in particular detachably, wherein it is preferred that the auxiliary element is adhesively connected to the base element.

11. The mold element according to claim 8 wherein the base element and/or the auxiliary element comprises TPU, PC, APET, PPC and/or PETG, in particular consists thereof.

12. The mold element according to claim 8 wherein the base element and/or the auxiliary element comprises a film, in particular consists thereof, wherein it is preferred that the base element has a thickness of 0.5-1.5 mm and/or the auxiliary element has a thickness of 0.1-2 mm.

13. The mold element according to claim 8 wherein the mold openings are cylindrical or conical, preferably with a round, triangular or quadrangular, particularly preferred square cross-section.

14. The mold element according to claim 8 wherein the auxiliary element comprises:
a higher hot forming temperature than the base element, and/or
a higher elasticity than the base element.

15. The mold element according to claim 8 wherein mold openings have on the first surface a cross-sectional area of 0.04 mm²-0.16 mm², in particular of 0.04-0.08 mm².

16. The mold element according to claim 8 wherein the mold openings have a depth of 600 μm to 2200 μm, in particular of 600 μm to 1000 μm.

17. The mold element according to claim 8 wherein the base element has a thickness of 500 to 1.5 mm and/or the auxiliary element has a thickness of 100 μm to 2 mm.

18. The mold element according to claim 8 wherein mold openings have on the second surface and/or on a surface of the auxiliary element a cross-sectional area of <1200 μm², in particular of <100 μm².
